(19) United States
(12) Patent Application Publication
(10) Pub. No.: US 2025/0177618 A1
(43) Pub. Date: Jun. 5, 2025

(54) Breast pump

(71) Applicant: Shenzhen Oceanwing Smart Innovations Technology Co., Ltd, Shenzhen (CN)

(72) Inventors: Liying Chen, Shenzhen (CN); Zhihai Zhang, Shenzhen (CN); Yingying Qiao, Shenzhen (CN)

(21) Appl. No.: 18/966,544

(22) Filed: Dec. 3, 2024

(30) Foreign Application Priority Data
Dec. 5, 2023 (CN) 202323320156.1

(51) Int. Cl. A61M 1/06

(52) U.S. Cl. CPC A61M 1/062; A61M 2205/33

(57) ABSTRACT

Disclosed is a breast pump comprising a milk bin and a cover. The cover includes a cover body and an arc-shaped heating device. The cover body is connected with the milk bin, and the arc-shaped heating device is disposed in the cover body. The cover body comprises a lower area and an upper area separated by a wall. The lower area is connected to the upper area in a height direction of the breast pump, and the lower area and the milk bin form a milk storage chamber. A ratio of a height of the arc-shaped heating device in the upper area to a total height of the arc-shaped heating device is greater than or equal to 0.7 in the height direction of the breast pump.

### CROSS-REFERENCE TO RELATED APPLICATION

The present disclosure claims priority of the Chinese Patent Application No. 202323116454.9, filed on Nov. 17, 2023 before the China National Intellectual Property Administration, entitled “Breast Pump”, which is incorporated herein by reference in its entirety.

### TECHNICAL FIELD

The present disclosure relates to the technical field of mother and baby products, in particular to a breast pump.

### BACKGROUND

With the development of technology, breast pumps on the market add a heating function successively. When a heating breast pump is used, a trumpet cover (e.g., a horn cover) of the breast pump can fit the breast and hot compress the breast, thereby improving use comfort of the breast pump and avoiding diseases or the like caused by engorgement as much as possible.

In the related art, when a wearable breast pump sucks breast milk in for storage, the breast milk contacts the trumpet cover, the present trumpet covers easily heat the breast milk in the breast pump a lot when the breast pump sucks the breast milk, and the breast milk in the breast pump is usually heated once before feeding the infant, thereby causing double heating of the breast milk. Double heating of breast milk may cause loss of nutrients in the breast milk, and even germinate bacteria and produce harmful substances.

### SUMMARY

The present disclosure describes a breast pump, which can achieve local heating of a cover (e.g., a trumpet cover, a housing), and can reduce heating of breast milk by an arc-shaped heating member during use of the breast pump, thereby reducing double heating of the breast milk by the breast pump, so as to reduce situations that the breast milk in the breast pump loses nutrients, germinates bacteria and produces harmful substances.

In a first aspect, an example of the disclosure provides a breast pump comprising a milk bin and a cover (e.g., a trumpet cover, a housing). The cover comprises a cover body and an arc-shaped heating member (e.g., heating device). The cover body is connected with the milk bin, and the arc-shaped heating member is disposed in the cover body. The cover body comprises a lower area and an upper area which are connected to each other in a height direction of the breast pump. The lower area and the upper area may be separated by wall. The lower area and the milk bin enclose a milk storage chamber. At least part of the arc-shaped heating member is located in the upper area. A ratio of a height of the arc-shaped heating member in the upper area to a total height of the arc-shaped heating member is greater than or equal to 0.7 in the height direction of the breast pump.

In some examples, the arc-shaped heating member comprises an arc-shaped section and an end section. The end section is connected to at least one end of the arc-shaped section and extends from the arc-shaped section. The arc-shaped section is disposed in the upper area, and the end section is located in the lower area. A ratio of a height of the arc-shaped section to the total height in the height direction of the breast pump is greater than or equal to 0.7.

In some examples, the number of the end sections is two, and the two end sections are connected to two ends of the arc-shaped section correspondingly. A ratio of an extension height of each of the end sections to the total height in the height direction of the breast pump is smaller than or equal to 0.3.

In some examples, the ratio of the height in the upper area to the total height of the arc-shaped heating member in the height direction of the breast pump is greater than or equal to 0.8 or 0.9.

In some examples, the milk bin comprises a top wall enclosing the milk storage chamber, and the upper area and the lower area are separated by a plane of the top wall.

In some examples, a part of the arc-shaped heating member is located in the lower area, and the part of the arc-shaped heating member located in the lower area has a first lowest position in the height direction of the breast pump. The milk bin comprises a bottom wall disposed opposite the top wall, and the bottom wall has a second lowest position in the height direction of the breast pump.

In some examples, a distance in the height direction between the first lowest position and the plane of the top wall is set as a first distance, a distance in the height direction between the second lowest position and the plane of the top wall is set as a second distance, and a ratio of the first distance to the second distance is smaller than 0.4.

In some examples, the cover body is provided with a liquid inlet channel extending along a preset axis and extends from the liquid inlet channel to a side thereof in a trumpet shape; the liquid inlet channel is in communication with the milk storage cavity, and the arc-shaped heating member is disposed around| the preset axis.

In some examples, the cover body comprises an annular fitting portion, a trumpet-shaped connecting portion, and a channel portion. The annular fitting portion, the trumpet-shaped connecting portion and the channel portion are connected in sequence. One end of the trumpet-shaped connecting portion having a larger circumferential size is connected to an inner ring of the annular fitting portion, and the other end of the trumpet-shaped connecting portion having a smaller circumferential size is connected to one end of the channel portion. The other end of the channel portion extends in a direction away from the trumpet-shaped connecting portion. The liquid inlet channel is provided in the channel portion. The annular fitting portion is configured to fit the human skin, and the arc-shaped heating member is disposed on the annular fitting portion.

In some examples, the cover body is integrally formed and wraps the arc-shaped heating member by integral forming, an outer periphery of the cover body is be provided with an embedding groove facing a middle area of the cover body, an outer periphery of the milk bin is provided with an embedding edge, and the embedding edge is embedded in the embedding groove, so that the cover body and the milk bin are tightly coupled.

In some examples, the breast pump comprises a host (e.g., a controller). The milk bin is provided with an accommodating area, and the host is accommodated in the accommodating area. The cover body comprises a first terminal assembly disposed on the arc-shaped heating member, the host is provided with a second terminal assembly, and the first terminal assembly is electrically connected to the second terminal assembly.

Beneficial effects of the present disclosure include: Different from situations in the related art, in the present disclosure, the arc-shaped heating member is provided in the cover, and in the height direction of the breast pump, at least most regions of the arc-shaped heating member may be located in the upper area of the trumpet cover, and only a few regions of the arc-shaped heating member may extend to the lower area of the trumpet cover. Alternatively, the arc-shaped heating member may be totally located in the upper area, and the lower area and the milk bin enclose the milk storage chamber. During use of the breast pump, the breast milk in the milk storage chamber is usually not higher than the lowest point of the arc-shaped heating member, which can reduce heating of the breast milk in the milk storage chamber by the arc-shaped heating member, and thus reduce a situation that the breast pump double heats the breast milk. This would reduce situations that the breast milk in the breast pump loses nutrients, germinates bacteria, and produces harmful substances.

### BRIEF DESCRIPTION OF DRAWINGS

FIG. 1 is a schematic diagram of an overall structure of a breast pump according to an example of the present disclosure;

FIG. 2 is a schematic diagram of an exploded structure of the breast pump shown in FIG. 1;

FIG. 3 is a schematic structural diagram of the breast pump shown in FIG. 1 cut along a section line A-A;

FIG. 4 is a schematic structural diagram of the breast pump shown in FIG. 1 from another viewing angle; and

FIG. 5 is another schematic diagram of an exploded structure of the breast pump shown in FIG. 1.

### DETAILED DESCRIPTION

Technical solutions in examples of the present disclosure will be clearly and fully described with reference to the accompanying drawings in the examples of the present disclosure. Obviously, the examples to be described are merely part of examples of the disclosure, but not all examples of the disclosure. Based on the examples of the disclosure, all other examples obtained by those of ordinary skill in the art without inventive work shall fall within the scope of the disclosure.

With the development of technology, breast pumps on the market add a heating function successively. When a heating breast pump is used, a trumpet cover of the breast pump can fit the breast and hot compress the breast, thereby improving use comfort of the breast pump and avoiding diseases or the like caused by engorgement as much as possible.

In the related art, when a wearable breast pump sucks breast milk in for storage, the breast milk contacts the trumpet cover, the present trumpet covers easily heat the breast milk in the breast pump a lot when the breast pump sucks the breast milk, and the breast milk in the breast pump is usually heated once before feeding the infant, thereby causing double heating of the breast milk. Double heating of breast milk may cause loss of nutrients in the breast milk, and even germinate bacteria and produce harmful substances. In order to solve the above problems, the present disclosure provides the following examples.

A breast pump 1 refers to a tool for extruding breast milk accumulated in a breast. The breast pump 1 may adhere to a breast of a human body, and can squeeze the breast or perform negative pressure suction on the breast, so as to extrude and collect breast milk in the breast.

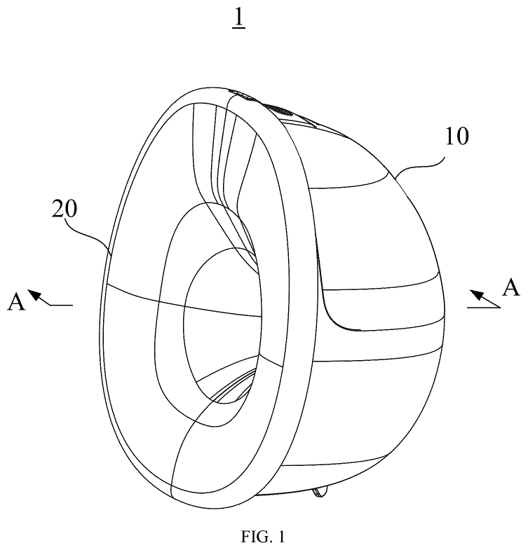

As shown in FIG. 1 to FIG. 3, the breast pump 1 may include a milk bin 10 and a trumpet cover 20, wherein the trumpet cover 20 may be configured to cover a nipple and an areola to fit a human breast and collect breast milk. The milk bin 10 can be connected with (e.g., hermetically connected with) the trumpet cover 20, and can receive breast milk flowing out of the trumpet cover 20 and store the breast milk. As shown in FIG. 1 to FIG. 3, the trumpet cover 20 may include a cover body 210 and a heating member 220 (e.g., a heating device such as electric pads). The heating member 220 may be in an arc-shape or curved shape. While the description herein refers to heating member 220 as an arc-shaped heating member 220. The shape of the heating member 220 is not limited to arc-shape. The cover body 210 may be hermetically connected with the milk bin 10, and the arc-shaped heating member 220 may be disposed inside the cover body 210. For example, the cover body 210 may adhere to the human body, and the arc-shaped heating member 220 can heat the breast of the human body through the cover body 210, so that the breast pump 1 can achieve a function of hot compress, and the arc-shaped heating member 220 can transfer heat which is milder and more uniform to skin of the human body through the cover body 210. In addition, the arc-shaped heating member 220 may be disposed inside the cover body 210, so that the cover body 210 can protect the arc-shaped heating member 220 and reduce exposure of the arc-shaped heating member 220, thereby improving waterproof and dustproof effects and improving electrical safety of the breast pump 1.

The arc-shaped heating member 220 may be, for example, a C-shaped structure, a U-shaped structure, or the like. Since a mother's breast is generally hemispherical or quasi-hemispherical in shape, the arc-shaped heating member 220 can adapt to the shape of the breast and can correspondingly provide a large heating area for the breast, so that the arc-shaped heating member 220 can better heat the breast of the human body, so as to improve a heating effect of the arc-shaped heating member 220.

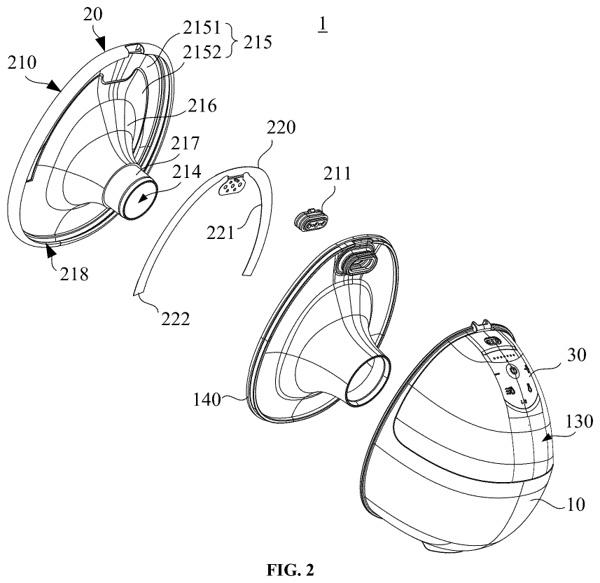

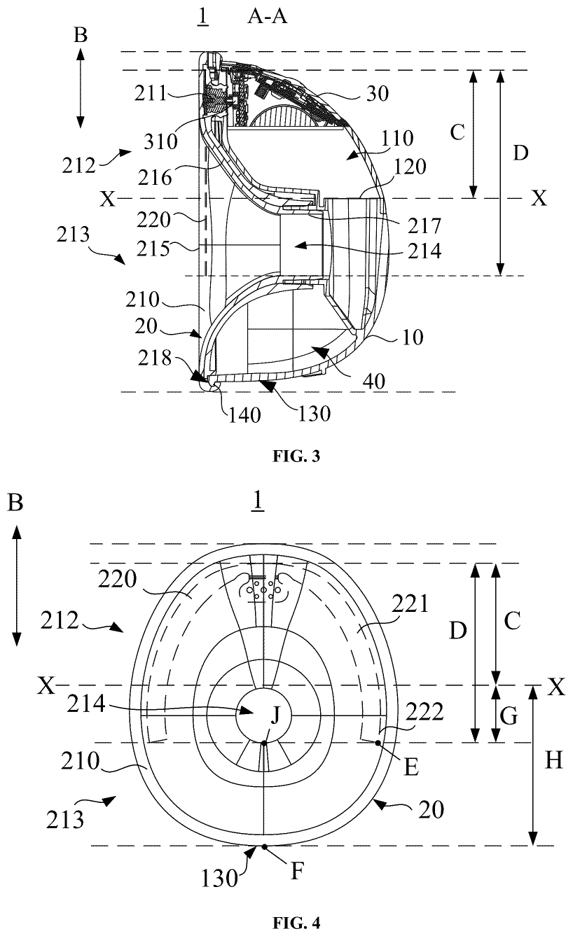

In some examples, as shown in FIG. 2 and FIG. 3, the breast pump 1 may further include a host 30 (e.g., a controller that comprise one or more processors), the milk bin 10 may be provided with an accommodating area 110, the host 30 may be accommodated in the accommodating area 110, and the host 30 may be connected to the milk bin 10 and the trumpet cover 20. The host 30 may be connected to the arc-shaped heating member 220 and control the arc-shaped heating member 220 to heat and control a temperature of the arc-shaped heating member 220. The host 30 may include a control element such as one or more circuit boards or processors, and the arc-shaped heating member 220 may be controlled by the control element.

As shown in FIG. 2 to FIG. 5, the cover body 210 may include a first terminal assembly 211 (e.g., a first connector) disposed on the arc-shaped heating member 220, the host 30 may be provided with a second terminal assembly 310 (e.g., a second connector), and the first terminal assembly 211 is electrically connected to the second terminal assembly 310. The host 30 is electrically connected to the arc-shaped heating member 220 through the first terminal assembly 211 and the second terminal assembly 310, so that the host 30 can control the arc-shaped heating member 220.

Specifically, after the trumpet cover 20 is connected and assembled with the milk bin 10 and the host 30, a sealed space can be formed at a joint between the first terminal assembly 211 and the second terminal assembly 310, so that liquid such as water and breast milk impurities such as dust are not prone to enter the joint between the first terminal assembly 211 and the second terminal assembly 310 to affect connection therebetween, and are not prone to enter an interior of the host 30 and an interior of the cover body 210 from the joint between the first terminal assembly 211 and the second terminal assembly 310, so as to protect the host 30 and the arc-shaped heating member 220 and improve electrical safety of the breast pump 1.

As shown in FIG. 3 to FIG. 4, the cover body 210 may include an upper area 212 and a lower area 213 connected to each other along a height direction of the breast pump 1. The lower area 213 and the milk bin 10 may enclose a milk storage chamber 40 configured to store breast milk. This can reduce the volume of the breast pump 1, so that the breast pump 1 occupies less space, and the structure of the breast pump 1 can be simplified.

In some examples, as shown in FIGS. 3 and 4, the milk bin 10 may include a top wall 120 (e.g., a partition) enclosing the milk storage chamber 40, and the upper area 212 and the lower area 213 may be separated by the top wall 120 (e.g., a plane X of the top wall 120). The top wall 120 of the milk storage chamber 40 may be set as an interface between the upper area 212 and the lower area 213, so that the breast milk in the milk storage chamber 40 is correspondingly in the lower area 213, so that the position of the breast milk in the breast pump 1 can be better distinguished.

At least part of the arc-shaped heating member 220 may be located in the upper area 212, and a ratio of a height of the arc-shaped heating member 220 in the upper area 212 to a total height of the arc-shaped heating member 220 may be greater than or equal to 0.7 in a height direction of the breast pump 1.

The height direction of the breast pump 1 may be as shown by an arrow B in FIG. 3 and FIG. 4, the height of the arc-shaped heating member 220 in the upper area 212 may be as shown in a height C in FIG. 3 and FIG. 4, and the total height of the arc-shaped heating member 220 may be as shown in a height D in FIG. 3 and FIG. 4, wherein C: D ≥0.7.

The ratio of the height in the upper area to the total height of the arc-shaped heating member 220 in the height direction of the breast pump 1 may be greater than or equal to 0.8, or 0.9. For example, the ratio of the height in the upper area to the total height of the arc-shaped heating member 220 in the height direction of the breast pump 1 may be 0.85, 0.95, or the like. In this way, most regions of the arc-shaped heating member 220 may be disposed in the upper area 212, and only a few regions extend to the lower area 213 of the trumpet cover 20, which can reduce a contact area between the arc-shaped heating member 220 and the lower area 213 of the trumpet cover 20, and can reduce heating of the breast milk in the milk storage chamber 40 by the arc-shaped heating member 220.

In other examples, the ratio of the height in the upper area to the total height of the arc-shaped heating member 220 may be equal to 1, (e.g., the arc-shaped heating member 220 is totally located in the upper area 212 to only heat the upper area, and it is not easy to heat milk in the milk storage chamber 40 corresponding to the lower area 213).

By disposing most or all of the arc-shaped heating member 220 in the upper area 212 of the cover body 210, the arc-shaped heating member 220 can achieve local heating, so that the arc-shaped heating member 220 can reduce heating of the lower area 213 during heating. The lower area 213 and the milk bin 10 may enclose (e.g., form) the milk storage chamber 40. The breast milk in the milk storage chamber 40 is usually not higher than the lowest point of the arc-shaped heating member 220 during use of the breast pump 1, which can reduce heating of the breast milk in the milk storage chamber 40 by the arc-shaped heating member 220, and thus reduce a situation that the breast pump 1 double heats the breast milk, so as to reduce situations that the breast milk in the breast pump 1 loses nutrients, germinates bacteria, or produces harmful substances.

As shown in FIG. 3, the accommodating area 110 may be disposed in the upper area 212 of the cover body 210, and both the host 30 and the first terminal assembly 211 may be connected to the upper area 212 and the arc-shaped heating member 220, so that the host 30 is connected to the arc-shaped heating member 220.

In some examples, as shown in FIG. 2 to FIG. 4, the arc-shaped heating member 220 may include an arc-shaped section 221 and an end section 222. The end section 222 is connected to at least one end of the arc-shaped section 221 and extends from the arc-shaped section 221. The arc-shaped section 221 is disposed in the upper area 212, and the end section 222 is located in the lower area 213. A ratio of a height of the arc-shaped section 221 to the total height in the height direction of the breast pump 1 is greater than or equal to 0.7.

The height of the arc-shaped section 221 in the height direction of the breast pump 1 may be as shown in the height C in FIG. 4.

A ratio of the height of the arc-shaped section 221 to the total height in the height direction of the breast pump 1 may be greater than or equal to 0.8, or 0.9. For example, the ratio of the height of the arc-shaped section 221 to the total height in the height direction of the breast pump 1 may be 0.85, 0.95, or the like.

In some examples, a ration of the height of the arc-shaped section 221 to the height of the arc-shaped heating member 220 in the height direction of the breast pump 1 may be 1. The arc-shaped heating member 220 is the arc-shaped section 221, and does not include an end section. The entire arc-shaped heating member 220 is disposed in the upper area 212 of the cover body 210.

The arc-shaped section 221 is disposed in the upper area 212, and a proportion of the arc-shaped section 221 in the arc-shaped heating member 220 is relatively large, so that most of the arc-shaped heating member 220 is located in the upper area 212 or may be totally located in the upper area 212, the heating of the arc-shaped heating member 220 is mainly concentrated in the upper area 212, thereby reducing the heating of the lower area 213 by the arc-shaped heating member 220 during the use of the breast pump 1, and reducing the heating of the breast milk in the milk storage chamber 40.

In some examples, the arc-shaped heating member 220 may comprise two end sections (e.g., the number of the end sections 222 may be two), and the two end sections 222 are connected to two ends of the arc-shaped section 221 respectively. This can make the heating area of the arc-shaped heating section more uniform, make the arc-shaped heating member 220 provide a large heating area to improve heating efficiency and heating effect of the breast.

A ratio of an extension height of each of the end sections 222 to the total height in the height direction of the breast pump 1 may be smaller than or equal to 0.3, smaller than or equal to 0.2, and smaller than or equal to 0.1.

The extension height of each of the end sections 222 in the height direction of the breast pump 1 may be set corresponding to a height of the arc-shaped section 221. For example, in the height direction of the breast pump 1, a ratio of the height of the arc-shaped section 221 to the extension height of each of the end sections 222 may be 7:3, 8:2, 9:1, or the like.

In some examples, as shown in FIG. 4, a part of the arc-shaped heating member 220 may be located in the lower area 213, and the part of the arc-shaped heating member 220 located in the lower area 213 may have a first lowest position in the height direction of the breast pump 1. The first lowest position may be at the end section 222, as shown in a position E in FIG. 4.

The milk bin 10 may include a bottom wall 130 disposed opposite the top wall 120, and the bottom wall 130 has a second lowest position in the height direction of the breast pump 1. The bottom wall 130 may be located at the bottom of the milk bin 10. The second lowest position may be as shown in a position F in FIG. 4.

A distance in the height direction between the first lowest position and the plane of the top wall 120 may be set as a first distance, a distance in the height direction between the second lowest position and the plane of the top wall 120 may be set as a second distance, and a ratio of the first distance to the second distance may be smaller than 0.4. The first distance may be as shown by a distance G in FIG. 4, and the second distance may be as shown by a distance H in FIG. 4, where G: H≤0.4.

In this way, a height of the arc-shaped heating member 220 extending to the lower area 213 is high, the lowest heating area of the arc-shaped heating member 220 is not easy to contact a liquid level of milk in the milk storage chamber 40, and the milk in the milk storage chamber 40 is not easy to be higher than the lowest point of the arc-shaped heating member 220, so as to reduce heating of the liquid level and the milk by the arc-shaped heating member 220, and reduce double heating of the breast milk by the breast pump 1.

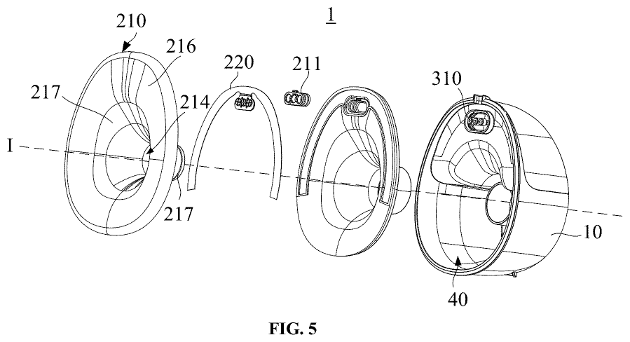

In some examples, as shown in FIG. 2 to FIG. 5, the cover body 210 may be provided with a liquid inlet channel 214 extending along a preset axis, and may extend from the liquid inlet channel 214 to a side thereof in a trumpet shape. The liquid inlet channel 214 may be in communication with the milk storage chamber 40, and the arc-shaped heating member 220 is disposed around| the preset axis. The preset axis may be as shown by I in FIG. 5.

In this way, the shape of the cover body 210 corresponds to the breast of the human body, the cover body 210 can cover a nipple and an areola to better fit the human breast and perform negative pressure suction on the breast, so that the cover body 210 is convenient to stimulate the nipple to produce milk and collect the breast milk. The milk may further enter the milk storage chamber 40 through the liquid inlet channel 214.

The arc-shaped heating member 220 is disposed around the preset axis, so that the shape of the heating member fit the shape of the cover body 210 and the shape of the breast, so as to enlarge the heating area and better achieve the hot compress function.

In some examples, as shown in FIG. 2 to FIG. 5, the cover body 210 may include an annular fitting portion 215, a trumpet-shaped connecting portion 216, and a channel portion 217.

The annular fitting portion 215, the trumpet-shaped connecting portion 216 and the channel portion 217 may be connected in sequence, one end of the trumpet-shaped connecting portion 216 having a larger circumferential size is connected to the annular fitting portion 215, the other end of the trumpet-shaped connecting portion 216 having a smaller circumferential size is connected to one end of the channel portion 217, and the other end of the channel portion 217 extends in a direction away from the trumpet-shaped connecting portion 216. The liquid inlet channel 214 is provided in the channel portion 217. The annular fitting portion 215 is configured to fit the human skin, and the arc-shaped heating member 220 is disposed on the annular fitting portion 215.

The trumpet-shaped connecting portion 216 and the channel portion 217 may correspond to the areola and nipple of a human body. When a user uses the breast pump 1 according to the disclosure, the nipple may be correspondingly placed in the channel portion 217, and the trumpet-shaped connecting portion 216 may correspondingly adhere to the areola of the human body. The trumpet-shaped connecting portion may fit the human body at or surround the areola, so that the breast pump 1 can better fit the human body and is not prone to fall off, and can form better negative pressure suction when the breast pump 1 operates.

The arc-shaped heating member 220 is disposed on the annular fitting portion 215, so that the arc-shaped heating member 220 can hot compress the position or the periphery of the areola of the human body by means of the annular fitting portion 215, thereby alleviating swelling and pain of the mother's breast when the breast pump 1 is used, and also improving milk yield and milk production speed of the mother.

In some examples, as shown in FIG. 4, the channel portion 217 may be disposed in the lower area 213 of the cover to communicate with the milk storage chamber 40. In the height direction of the breast pump 1, the lowest position of the liquid inlet channel 214 may be aligned with the first lowest position of the arc-shaped heating member 220, or the lowest position of the liquid inlet channel 214 may be lower than the first lowest position of the arc-shaped heating member 220. The lowest position of the liquid inlet channel 214 may be as shown by a position J in FIG. 4.

When the breast pump 1 collects breast milk, if the milk in the milk storage chamber 40 is higher than the lowest position of the liquid inlet channel 214, the milk flows out of the milk storage chamber 40 through the liquid inlet channel 214, so that when the breast pump 1 is placed level along the height direction thereof, a highest liquid level in the milk storage chamber 40 is lower than the lowest position of the liquid inlet channel 214. Therefore, the first lowest position of the arc-shaped heating member 220 may be set to be higher than or flush with the lowest position of the liquid inlet channel 214, so that heating of the milk in the milk storage chamber 40 by the arc-shaped heating member 220 can be reduced, and the heating area of the arc-shaped heating member 220 can be enlarged.

At least part of an outer peripheral wall of the channel portion 217 is exposed to the milk storage chamber 40, so that the breast milk in the liquid inlet channel 214 of the channel portion 217 can enter the milk storage chamber 40.

In some examples, as shown in FIG. 2 to FIG. 5, the arc-shaped heating member 220 may surround an inner ring 2152 of the annular fitting portion 215, and among the inner ring 2152 and an outer ring 2151 of the annular fitting portion 215, the arc-shaped heating member 220 may be closer to the outer ring 2151 of the annular fitting portion 215.

Further, both the inner ring 2152 and the outer ring 2151 of the annular fitting portion 215 may be annularly disposed around the preset axis and connected to each other, the inner ring 2152 of the annular fitting portion 215 is correspondingly connected to the trumpet-shaped connecting portion 216, and the outer ring 2151 extends outward from the inner ring 2152.

Since both the inner ring 2152 and the outer ring 2151 of the annular fitting portion 215 may be annularly disposed around the preset axis, the arc-shaped heating member 220 may be disposed on the more peripheral outer ring 2152, so that the arc-shaped heating member 220 has a longer extension length and a larger size, thereby enlarging the heating area of the arc-shaped heating member 220 and improving the heating effect of the arc-shaped heating member 220.

In some examples, as shown in FIG. 2 to FIG. 3, the cover body 210 may be integrally formed and wraps around the arc-shaped heating member 220 by integral forming. An outer periphery of the cover body 210 may be provided with an embedding groove 218 facing a middle area of the cover body 210. An outer periphery of the milk bin 10 may comprise an embedding edge 140, and the embedding edge 140 may be embedded in the embedding groove 218, so that the cover body 210 and the milk bin 10 are tightly coupled.

The cover body 210 may be formed by injection molding. The cover body 210 can wrap the arc-shaped heating member 220 therein during injection molding, so that the arc-shaped heating member 220 tightly adheres to the cover body 210, and difficulty in preparation of the trumpet cover 20 can also be reduced.

Due to advantages of high forming speed and accurate product size, injection molding may be used, so that the size of the cover body 210 is more accurate and the cover body 210 can be better tightly coupled to the milk bin 10.

The cover body 210 may be soft silica gel, so that the trumpet cover 20 can be adapted to different breast shapes to tightly adhere to the breast, and the breast pump 1 performs negative pressure suction on the breast. Moreover, since the soft silica gel has certain elasticity, when the embedding edge 140 on the outer periphery of the milk bin 10 is embedded into the embedding groove 218 of the cover body 210, the embedding groove 218 can tightly adhere to the embedding edge 140, so that the cover body 210 and the milk bin 10 enhance the sealing effect while achieving stable coupling.

Due to the tight coupling between the cover body 210 and the milk bin 10, liquid such as moisture or impurities such as dust are not prone to enter the cover body 210 and the milk bin 10 to pollute the breast milk or damage the host 30 and the arc-shaped heating member 220.

In the present disclosure, the arc-shaped heating member 220 is provided in the trumpet cover 20, and in the height direction of the breast pump 1, most regions of the arc-shaped heating member 220 is located in the upper area 212 of the trumpet cover 20, and only a few regions of the arc-shaped heating member 220 extends to the lower area 213 of the trumpet cover 20. Alternatively, the arc-shaped heating member 220 may be totally located in the upper area 212, and the lower area 213 and the milk bin 10 enclose the milk storage chamber 40. During use of the breast pump 1, the breast milk in the milk storage chamber 40 is usually not higher than the lowest point of the arc-shaped heating member 220, which can reduce heating of the breast milk in the milk storage chamber 40 by the arc-shaped heating member 220, and thus reduce a situation that the breast pump 1 double heats the breast milk, so as to reduce situations that the breast milk in the breast pump 1 loses nutrients, germinates bacteria or, produces harmful substances.

The above are only examples of the present disclosure, and do not limit the scope of the disclosure. Any equivalent structure or process transformation made by using the contents of description and drawings of the present disclosure, or direct or indirect use thereof in other related technical fields should be included in the scope of the present disclosure.

What is claimed is:

1. A breast pump comprising:
a milk bin; and
a cover comprising a cover body and an arc-shaped heating device, wherein the cover body is connected to the milk bin, wherein:
the cover body comprises a lower area and an upper area separated by a wall,
the lower area is connected to the upper area in a height direction of the breast pump,
the lower area and the milk bin form a milk storage chamber, and
a ratio of a height of the arc-shaped heating device in the upper area to a total height of the arc-shaped heating device is greater than or equal to 0.7 in the height direction of the breast pump.

2. The breast pump according to claim 1, wherein:
the arc-shaped heating device comprises an arc-shaped section and an end section,
the end section is connected to at least one end of the arc-shaped section and extends from the arc-shaped section,
the arc-shaped section is located in the upper area, and the end section is located in the lower area, and
a ratio of a height of the arc-shaped section to a total height in the height direction of the breast pump is greater than or equal to 0.7.

3. The breast pump according to claim 2, wherein:
the arc-shaped heating device comprises two end sections connected to two ends of the arc-shaped section correspondingly, and
a ratio of an extension height of each of the two end sections to the total height in the height direction of the breast pump is smaller than or equal to 0.3.

4. The breast pump according to claim 1, wherein the ratio of the height of the arc-shaped heating device in the upper area to the total height of the arc-shaped heating device in the height direction of the breast pump is greater than or equal to 0.8 or 0.9.

5. The breast pump according to claim 1, wherein the milk bin comprises the wall enclosing the milk storage chamber.

6. The breast pump according to claim 5, wherein:
a part of the arc-shaped heating device is located in the lower area, and the part of the arc-shaped heating device located in the lower area has a first lowest position in the height direction of the breast pump,
the milk bin comprises a bottom wall disposed opposite the wall, and the bottom wall has a second lowest position in the height direction of the breast pump, and
a ratio of a distance in the height direction between the first lowest position and a plane of the wall to a distance in the height direction between the second lowest position and the plane of the wall is smaller than 0.4.

7. The breast pump according to claim 1, wherein:
the cover body comprises a liquid inlet channel extending along a preset axis, and
the liquid inlet channel is connected to the milk storage chamber, and
the arc-shaped heating device is disposed around the preset axis.

8. The breast pump according to claim 7, wherein:
the cover body comprises an annular fitting portion, a trumpet-shaped connecting portion, and a channel portion,
the annular fitting portion, the trumpet-shaped connecting portion, and the channel portion are connected in sequence,
one end of the trumpet-shaped connecting portion having a larger circumferential size is connected to an inner ring of the annular fitting portion,
another end of the trumpet-shaped connecting portion having a smaller circumferential size is connected to one end of the channel portion,
another end of the channel portion extends in a direction away from the trumpet-shaped connecting portion,
the channel portion comprises the liquid inlet channel, and
the arc-shaped heating device is disposed on the annular fitting portion.

9. The breast pump according to claim 1, wherein:
the cover body wraps around the arc-shaped heating device by integral forming,
an outer periphery of the cover body comprises an embedding groove facing a middle area of the cover body,
an outer periphery of the milk bin is provided with an embedding edge, and
the embedding edge is embedded in the embedding groove.

10. The breast pump according to claim 1, further comprising:
a controller, wherein:
the cover body comprises a first connector disposed on the arc-shaped heating device, and
the controller comprises a second connector electrically connected to the first connector.

11. A breast pump comprising:
a milk bin;
a cover comprising a cover body and a heating device, the cover body being connected to the milk bin, the cover body comprising a lower area and an upper area separated by a wall, the heating device being located in the upper area of the cover body; and
a controller configured to control operation of the heating device.

12. The breast pump according to claim 11, wherein the heating device is in the shape of an arc.

13. The breast pump according to claim 11, wherein the heating device is on an edge of the cover.

14. The breast pump according to claim 11, wherein the heating device comprises electric pads controller by the controller.

15. The breast pump according to claim 11, further comprising a milk storage chamber, wherein the milk bin comprises the wall enclosing the milk storage chamber.

16. The breast pump according to claim 11, wherein:
the cover body comprises a liquid inlet channel extending along a preset axis, and
the liquid inlet channel is connected to the milk bin, and
the heating device is disposed around the preset axis.

17. The breast pump according to claim 11, wherein:
an outer periphery of the cover body comprises an embedding groove facing a middle area of the cover body,
an outer periphery of the milk bin is provided with an embedding edge, and
the embedding edge is embedded in the embedding groove.

18. The breast pump according to claim 11, wherein the cover body comprises a first connector disposed on the heating device, and the controller comprises a second connector electrically connected to the first connector.

19. A breast pump comprising:
a milk bin;
a housing connected to the milk bin, wherein the housing is divided into a lower area and an upper area by a wall; and
a heating device in the upper area of the housing; and
a controller configured to control operation of the heating device.

20. The breast pump according to claim 19, wherein the heating device is in the shape of an arc and is positioned inside the housing.
